(19) United States
(12) Patent Application Publication
(10) Pub. No.: US 2014/0341803 A1
(43) Pub. Date: Nov. 20, 2014

(54) STABLE NANOEMULSIONS USEFUL IN THE TREATMENT OF CANCER

(71) Applicant: Natalya Rapoport, Sandy, UT (US)

(72) Inventor: Natalya Rapoport, Sandy, UT (US)

(73) Assignee: UNIVERSITY OF UTAH RESEARCH FOUNDATION, Salt Lake City, UT (US)

(21) Appl. No.: 14/277,405

(22) Filed: May 14, 2014

Related U.S. Application Data
(60) Provisional application No. 61/824,474, filed on May 17, 2013.

(51) Int. Cl. A61K 9/107; A61K 47/34; A61K 31/337; A61K 47/08

(52) U.S. Cl. CPC A61K 9/1075; A61K 47/08; A61K 47/34; A61K 31/337
USPC 424/1.61; 514/772.1; 514/449

(57) ABSTRACT

Described herein are methods for treating a tumor or cancer in a subject. The methods involve administering to the subject a nanoemulsion comprising (1) at least one perfluoro crown ether and (2) a block copolymer comprising a hydrophilic block and hydrophobic block, wherein the hydrophobic block is poly(d,l)lactic acid, and wherein the nanoemulsion comprises a therapeutic agent encapsulated in the nanoemulsion. The methods do not require the application of ultrasound or other sources of radiation in order to treat a tumor or cancer in the subject.

### CROSS REFERENCE TO RELATED APPLICATIONS

This application claims priority upon U.S. provisional application Ser. No. 61/824,474, filed on May 17, 2013. The application is hereby incorporated by reference in its entirety for all of its teachings.

### ACKNOWLEDGEMENT

The research leading to this invention was funded in part by the national Institutes of Health, Grant No. R01 EB1033. The Government has certain rights in this invention.

### BACKGROUND

Chemotherapy remains the treatment of choice for many types of cancer. During the last decade, progress in nanotechnology has enabled tumor-targeted delivery of anticancer drugs, which simultaneously decreased side effects and increased drug concentration in tumor tissue. The goal is to have an agent that exclusively targets the tumor. In pursuit of this goal, a number of groups have been directing their efforts to increase the degree of drug tumor-targeting using ultrasound-mediated drug delivery. In this approach, drug delivery with nanoparticles is combined with tumor-directed ultrasound that affects both the drug carrier and tumor tissue. The use of ultrasound triggers drug release from the nanoparticle carrier and increases drug and carrier extravasation and deposition in tumor cells.

Ultrasound may exert both positive and negative effects on biological tissue. Positive effects may be related to increased drug carrier and drug extravasation, drug release from carrier and drug internalization by tumor cells. On the other hand, vasodilation or vasoconstriction in response to ultrasound and cavitating microbubbles may result in cellular response of surrounding tissues such as inflammation, edema, hemorrhage, which could be negative. Thus, it would be desirable to treat cancer using chemotherapy that did not require the use of ultrasound or other sources of radiation.

### SUMMARY

Described herein are methods for treating a tumor or cancer in a subject. The methods involve administering to the subject a nanoemulsion comprising (1) at least one perfluoro crown ether and (2) a block copolymer comprising a hydrophilic block and hydrophobic block, wherein the hydrophobic block is poly(d,l)lactic acid, and wherein the nanoemulsion comprises a therapeutic agent encapsulated in the nanoemulsion. The methods do not require the application of ultrasound or other sources of radiation in order to treat a tumor or cancer in the subject.

The advantages of the invention will be set forth in part in the description which follows, and in part will be obvious from the description, or may be learned by practice of the aspects described below. The advantages described below will be realized and attained by means of the elements and combinations particularly pointed out in the appended claims. It is to be understood that both the foregoing general description and the following detailed description are exemplary and explanatory only and are not restrictive.

### BRIEF DESCRIPTION OF THE DRAWINGS

The accompanying drawings, which are incorporated in and constitute a part of this specification, illustrate several aspects described below.

FIG. 1 shows an example of a nanoparticle size distribution for 5% PEG-PDLA/1% PFCE formulation; 50-nm particles are residual micelles; 262 nm particles are nanodroplets. Nanodroplet size may be decreased by increasing sonication pressure during emulsification. Micelle fraction can be decreased by decreasing copolymer concentration and/or increasing PFCE concentration (50). PTX loading slightly increases nanodroplet sizes (e.g. from 260 to 280 nm).

FIG. 2 shows (A) schematic representation of the mouse positioning on the small animal MRgFUS device; and (B) an axial image of mouse 59 on the small animal MRgFUS device with labeled transducer and agar holder. The white arrow indicates the tumor (initial size, 455 mm³).

FIG. 3 shows photographs (A, C) and whole-body fluorescence images (B, D) of a mouse before (A, B) and after (C, D) combined treatment with PTX-loaded nanodroplets and MRgFUS. The dashed circles in (B,D) indicate the tumor location. Treatment parameters: MRgFUS was applied 8 hours after drug injection; spiral beam pattern (5 mm diameter) shown in FIG. 3A; FUS at 3.1 MPa; sonication time 3 minutes. The tumor did not recur during a 5-month observation. The former location of the tumor is still slightly visible in D, indicated by the dashed white circle.

FIG. 4 shows THE temperature rise for 3 individual voxels indicated in the treatment path is shown.

FIG. 5 shows tumor growth curves for control (N=3, squares); tumors treated with PTX-loaded nanodroplets without ultrasound (N=7, triangles); and the best results for tumors treated with PTX-loaded nanodroplets and MRgFUS (N=4, diamonds).

FIG. 6 shows the effect of ultrasound pressure on the tumor growth curves in the presence of PTX-loaded nanodroplets for no MRgFUS (N=7, diamonds); MRgFUS at 4.2 MPa (N=3, triangles); MRgFUS at 4.8 MPa (N=2, squares).

FIG. 7 photographs of a mouse bearing two ovarian carcinoma tumors (A) immediately before and (B) three weeks after treatment. A mouse was treated by four systemic injections of a nanoemulsion composed of PEG-PLLA and PTX (20 mg/kg as PTX) given twice weekly. The right tumor was sonicated by 1 MHz CW ultrasound (nominal output) power density 3.4 W/cm², exposure duration 1 minute) delivered four hours after the injection of the nanoemulsion.

### DETAILED DESCRIPTION

Before the present compounds, compositions, and/or methods are disclosed and described, it is to be understood that the aspects described below are not limited to specific compounds, synthetic methods, or uses as such may, of course, vary. It is also to be understood that the terminology used herein is for the purpose of describing particular aspects only and is not intended to be limiting.

In this specification and in the claims that follow, reference will be made to a number of terms that shall be defined to have the following meanings:

It must be noted that, as used in the specification and the appended claims, the singular forms “a,” “an” and “the” include plural referents unless the context clearly dictates otherwise. Thus, for example, reference to “a therapeutic agent” includes two or more such therapeutic agents, and the like.

“Optional” or “optionally” means that the subsequently described event or circumstance can or cannot occur, and that the description includes instances where the event or circumstance occurs and instances where it does not. For example, the phrase “optionally a second therapeutic agent” means that the second therapeutic agent may or may not be present in the compositions used for the methods described herein.

“Treating” or “treatment” is meant the medical management of a patient with the intent to cure, ameliorate, stabilize, or prevent a disease, pathological condition, or disorder. This term includes active treatment, that is, treatment directed specifically toward the improvement of a disease, pathological condition, or disorder, and also includes causal treatment, that is, treatment directed toward removal of the cause of the associated disease, pathological condition, or disorder. In addition, this term includes palliative treatment, that is, treatment designed for the relief of symptoms rather than the curing of the disease, pathological condition, or disorder; preventative treatment, that is, treatment directed to minimizing or partially or completely inhibiting the development of the associated disease, pathological condition, or disorder; and supportive treatment, that is, treatment employed to supplement another specific therapy directed toward the improvement of the associated disease, pathological condition, or disorder.

“Therapeutic agent” refers to a chemical compound, a hormone, or a biological molecule including nucleic acids, peptides, proteins, and antibodies that can be used to treat a pre-existing condition or reduce the symptoms of the condition.

“Nanoemulsion” refers either to nanodroplets that are less than 1500 nm, or more preferably less than 1000 nm in diameter, which are capable of encapsulating a therapeutic agent.

“Subject” refers to mammals including, but not limited to, humans, non-human primates, sheep, dogs, rodents (e.g., mouse, rat, etc.), guinea pigs, cats, rabbits, cows, and non-mammals including chickens, amphibians, and reptiles, who are at risk for or have been diagnosed with a tumor and benefits from the methods and compositions described herein.

Concentrations, amounts, and other numerical data may be expressed or presented herein in a range format. It is to be understood that such a range format is used merely for convenience and brevity and thus should be interpreted flexibly to include not only the numerical values explicitly recited as the limits of the range, but also to include all the individual numerical values or sub-ranges encompassed within the ranges as if each numerical value and sub-range is explicitly recited. As an illustration, a numerical range of “about 1 to 5” should be interpreted to include not only the explicitly recited values of about 1 to about 5, but also include individual values and sub-ranges within the indicated range. Thus, included in this numerical range are individual values such as 2, 3, and 4 and sub-ranges such as from 1-3, from 2-4, and from 3-5, etc. as well as 1, 2, 3, 4, and 5, individually. The same principle applies to ranges reciting only one numerical value as a minimum or a maximum. Furthermore, such an interpretation should apply regardless of the breadth of the range or the characteristics being described.

It is understood that any given particular aspect of the disclosed compositions and methods can be easily compared to the specific examples and embodiments disclosed herein. By performing such a comparison, the relative efficacy of each particular embodiment can be easily determined Particularly preferred compositions and methods are disclosed in the Examples herein, and it is understood that these compositions and methods, while not necessarily limiting, can be performed with any of the compositions and methods disclosed herein.

Described herein are methods for treating a tumor or cancer in a subject. The methods involve administering to the subject a nanoemulsion comprising (1) at least one perfluoro crown ether and (2) a block copolymer comprising a hydrophilic block and hydrophobic block, wherein the hydrophobic block is poly(d,l)lactic acid, and wherein the nanoemulsion comprises a therapeutic agent encapsulated in the nanoemulsion. Each component is discussed in detail below.

The nanoemulsions useful herein include a perfluoro crown ether. Crown ethers are heterocyclic chemical compounds that are composed of a ring containing several ether groups. The most common crown ethers are oligomers of perfluoro ethylene oxide the repeating unit being perfluoro ethyleneoxy, i.e., —CF₂CF₂O—. However, other perfluoro alkylene oxides can be present in the crown ether including, but not limited to, perfluoro propylene oxide, perfluoro butylene oxide, and the like. Examples of this series of compounds are the tetramer (n=4), the pentamer (n=5), the hexamer (n=6), the heptamer (n=7), the octamer (n=8), the decamer (n=10), and the like. The perfluoro crown ether has at least 16 symmetrical fluorine atoms. In other aspects, perfluoro crown ether is any crown ether with all hydrogen atoms substituted with fluorine atoms. Examples of perfluoro crown ethers include, but are not limited to, perfluoro 12-crown-4 ether, perfluoro 15-crown-5 ether, perfluoro 18-crown-6 ether, perfluoro 21-crown-7 ether, perfluoro dibenzo-18-crown-6 ether, perfluoro diaza-18-crown-6 ether, or any combination thereof.

In certain aspects when imaging of the tumor is to be performed, it is desirable that the perfluoro crown ether be symmetrical. When the fluoro groups are symmetrical, the ¹⁹F MR spectrum is narrow and, thus, useful in imaging the biodistribution of a nanoemulsion. The number of fluorine atoms present in the perfluoro crown ether can also determine the intensity of the signal in the ¹⁹F MR spectrum. In one aspect, the perfluoro crown ether has at least 16 fluorine atoms, at least 18 fluorine atoms, or at least 20 fluorine atoms.

Turning to the block copolymer, it includes a hydrophilic block and a hydrophobic block. In one aspect, the hydrophilic block can include a poly(alkylene oxide), a polyvinyl polymer such as polyvinyl pyrrolidone, or any combination thereof. In certain aspects, the hydrophilic block includes a poly(alkylene oxide). In some aspects, the poly(alkylene oxide) can have a molecular weight ranging from 500 to 10,000 Da, from 1,000 to 8,000 Da, from 1,500 to 5,000 Da, or from 1,500 to 2,500 Da. For example, the poly(alkylene oxide) can include a polyethylene oxide, a polypropylene oxide, a polybutylene oxide, a polypentylene oxide, or a combination thereof. In another aspect, the poly(alkylene oxide) is a triblock copolymer such as PEO-PPO-PEO or PPO-PEO-PPO. In one aspect, the poly(alkylene oxide) is polyethylene oxide having a molecular weight of 1000 Da, 2000 Da, 3000 Da, 4000 Da, or 5000 Da.

In some aspects, the hydrophobic block is poly(d,l)lactic acid having a molecular weight ranging from 500 to 1000 Da, from 500 to 1500 Da, from 500 to 2000 Da, from 500 to 2500 Da, from 500 to 3000 Da, from 500 to 3500 Da, from 500 to 4000 Da, from 500 to 4500 Da, from 500 to 5000 Da, from 500 to 5500 Da, from 500 to 6000 Da, from 500 to 6500 Da, from 500 to 7000 Da, from 500 to 7500 Da, from 500 to 8000 Da, from 500 to 8500 Da, from 500 to 9000 Da, from 500 to 9500 Da, from 500 to 10000 Da, from 500 to 10500 Da, from 500 to 11000 Da, from 500 to 11500 Da, or from 500 to 12000 Da.

In some aspects, the nanoemulsions include nanosized micelles and nanodroplets that have diameters that are less than about 1500 nm, about 1400 nm, about 1300 nm, about 1200 nm, about 1100 nm, about 1050 nm, about 1000 nm, about 950 nm, about 900 nm, about 850 nm, about 800 nm, about 750 nm, about 700 nm, about 650 nm, about 600 nm, about 550 nm, about 500 nm, about 450 nm, about 400 nm, about 350 nm, about 300 nm, about 250 nm, about 200 nm, about 150 nm, about 100 nm, about 90 nm, about 80 nm, about 70 nm, about 60 nm, about 50 nm, about 40 nm, about 30 nm, about 20 nm, or about 10 nm.

The nanoemulsions described herein are compositions having a hydrophilic outer shell composed of the hydrophilic block of the block copolymer, and lipophilic inner shell composed on the hydrophobic block of the block copolymer, and a lipophilic inner core composed of the fluoro ether. The nanoemulsions make it possible to efficiently transport lipophilic therapeutic agents or drugs to tumors. Due to the defective tumor's vasculature, the nanoemulsions can be extravasated into the tumor. In one aspect, at least one therapeutic agent is encapsulated within the lipophilic core of the nanoemulsion. In some aspects, the therapeutic agent can include lipophilic drugs that have a low aqueous solubility. For example, these therapeutic agents can include chemotherapeutic drugs, hormones, or any other biologically or chemically active drugs, which include nucleic acids, peptides, proteins, and/or antibodies, that can be used to treat a condition such as various tumors and cancers. In some aspects, the therapeutic agent can include, but is not limited to, paclitaxel, doxorubicin, adriamycin, cisplatin, taxol, methotrexate, 5-fluorouracil, betulinic acid, amphotericin B, diazepam, nystatin, propofol, testosterone, estrogen, prednisolone, prednisone, 2,3-mercaptopropanol, progesterone, multiple drug resistant (MDR) suppressing agents, or any combination thereof. In some aspects, the therapeutic agent can include, but is not limited to, paclitaxel, doxorubicin, or any combination thereof. For example, in one aspect the therapeutic agent encapsulated in the nanoemulsion can include only paclitaxel. In some aspects, the therapeutic agent encapsulated in the nanoemulsion includes at least paclitaxel. In another aspect, the therapeutic agent encapsulated in the nanoemulsion can include only doxorubicin. In some aspects, the therapeutic agent encapsulated in the nanoemulsion includes at least doxorubicin. In some aspects, the therapeutic agent encapsulated in the nanoemulsion includes at least paclitaxel and doxorubicin. In other aspects, the block copolymer is poly(ethylene oxide)-co-poly(d,l-lactide), the perfluoro crown ether is perfluoro 15-crown-5 ether, and the therapeutic agent is paclitaxel.

The nanoemulsions described herein can also be modified to include a targeting moiety. Such targeting moieties can be advantageously used to target specific tissues and cells. In certain aspects, the nanoemulsions are modified on the hydrophilic outer surface of the nanoemulsion to include the targeting moiety. In certain aspects, the nanoemulsions are modified on the hydrophilic outer surface of the nanoemulsion to include the targeting moiety by incorporating PEG-phospholipids into the block copolymer nanodroplet shell. The targeting moiety may include a ligand specific for particular tumors or a ligand that is capable of targeting tumor tissue without damaging normal, non-tumor tissue. In one aspect, the targeting moiety, which includes but is not limited to a target ligand, assists the nanoemulsion in finding the targeted cells or tissue. A ligand may be any compound of interest which will bind to another compound, such as a receptor.

The preparation of nanoemulsions loaded with one or more therapeutic agents do not involve special handling and techniques. In one aspect, the block copolymer and therapeutic agent are dissolved in a solvent. Examples of solvents useful herein include, but are not limited to, dimethyl sulfoxide (DMSO), tetrahydrorfuran (THF), or dioxane. The amount of therapeutic agent that can be encapsulated in the nanoemulsion can vary. In one aspect, the therapeutic agent can be from 0.1 wt % to 10 wt %. In some aspects, the block copolymer can be from 0.1 wt % to 5 wt %. In one aspect, in the next step the solvent is evaporated and saline is added or the organic solution is dialyzed against saline. Next, the perfluoro crown ether is added and the mixture is emulsified by sonication. In some aspects, the perfluoro crown ether is from 0.1 vol % to 20 vol % relative to the nanoemulsion volume. Exemplary methods for making the nanoemulsions loaded with a therapeutic agent are provided in the Examples below.

The methods described herein present an efficient and passive targeting chemotherapeutic modality for solid tumors. The methods of treating tumors as described herein can be performed by contacting the tumor with a therapeutic agent encapsulated in a nanoemulsion without the need for exposing the tumor to ultrasonic radiation or some other for of radiation. In some aspects, the tumor can include a multidrug resistant tumor, an inoperable tumor, or a combination thereof. In certain aspects, the tumor includes, but is not limited to, breast cancer, pancreatic cancer, ovarian cancer, prostate cancer, colon cancer or a combination thereof. In one aspect, the methods described herein can reduce or prevent tumor growth of a tumor having a size of 10 mm³ to 1,000 mm³, 100 mm³ to 750 mm³, or 200 mm³ to 600 mm³.

As discussed above, ultrasound may exert negative effects on biological tissue. For example, vasodilation or vasoconstriction in response to ultrasound and cavitating microbubbles may result in cellular response of surrounding tissues such as inflammation, edema, hemorrhage, which could be negative. Examples of ultrasonic radiation used in the art include focused ultrasound (FUS) radiation, unfocused ultrasound, continuous wave (CW) ultrasound radiation, or pulsed waved (PW) ultrasound radiation. As demonstrated in the Examples, the use of ultrasound at particular frequencies can result in inflammation and burning of the skin. Furthermore, the Examples demonstrate that seven of a total of 51 mice treated with magnetic resonance-guided focused ultrasound (MRgFUS) died within several days of MRgFUS treatment. Conversely, no mice were killed when administered the nanoemulsion without MRgFUS treatment. Thus, the methods described herein provide a safer approach to reducing or preventing tumor growth without the need of the application of ultrasonic radiation or energy.

The nanoemulsions described herein can be administered to a subject multiple times in order to reduce pr prevent the growth of a tumor. As demonstrated in the Examples, a tumor treated with a nanoemulsion and exposed to MRgFUS regressed quickly and was not visible to the naked eye. However, tumor re-growth started 6 weeks after treatment. The recurrent tumor responded to a second treatment (administration of nanoemulsion without MRgFUS). In this example, the pancreatic tumor cells did not develop drug resistance.

The tumor can be contacted with a first nanoemulsion by direct injection into the tumor, by subcutaneous injection, by intramuscular injection, or via systemic injection of the nanoemulsion, which includes intravenous injection. When the nanoemulsion is administered systemically, adequate time is given for the nanoemulsion to extravasate into the tumor. In some aspects, a time ranging from about 4 hours to about 24 hours is provided to allow the nanoemulsion to extravasate into the tumor. In yet another aspect, a time ranging from about 4 hours to about 8 hours, from about 8 hours to about 14 hours or from about 10 hours to about 24 hours is provided to allow the nanoemulsion to extravasate into the tumor.

In certain aspects, the tumor can be contacted with a second nanoemulsion following contacting the tumor with a first nanoemulsion by the techniques described above. The first nanoemulsion and the second nanoemulsion can be the same or different (e.g., different therapeutic agent).

The nanoemulsions described herein are stable and can be stored for extended periods of time. For example, the nanoemulsuions with the therapeutic agent can be stored in a refrigerator for several days. This is an important advantage, as it permits storage of the nanoemulsion for repeated administration to the subject. Because of the pharmacodynamics and pharmacokinetics of these nanoemulsions, they can be advantageously used as imaging agents. In one aspect, imaging of a tissue contacted with the nanoemulsions described herein can be conducted by using ¹⁹F MRI. For example, when the nanoemulsion contains perfluoro 15-crown-5 ether (PFCE), ¹⁹F MRI can be used to image the presence of the nanoemulsion in the subject after administration. In particular, because 20 equivalent fluorine atoms are present in this example (i.e., perfluoro 15-crown-5 ether or PFCE), it is possible to monitor nanoemulsion distribution within a tumor and normal tissue.

The nanoemulsions described above can be administered to a subject using techniques known in the art. For example, pharmaceutical compositions can be prepared with the nanoemulsions. It will be appreciated that the actual preferred amounts of the nanoemulsion in a specified case will vary according to the specific nanoemulsions being utilized, the particular compositions formulated, the mode of application, and the particular situs and subject being treated. Dosages for a given host can be determined using conventional considerations, e.g. by customary comparison of the differential activities of the subject compounds and of a known agent, e.g., by means of an appropriate conventional pharmacological protocol. Physicians and formulators, skilled in the art of determining doses of pharmaceutical compounds, will have no problems determining dose according to standard recommendations (Physicians Desk Reference, Barnhart Publishing (1999).

Pharmaceutical compositions described herein can be formulated in any excipient the biological system or entity can tolerate. Examples of such excipients include, but are not limited to, water, saline, Ringer's solution, dextrose solution, Hank's solution, and other aqueous physiologically balanced salt solutions. Nonaqueous vehicles, such as fixed oils, vegetable oils such as olive oil and sesame oil, triglycerides, propylene glycol, polyethylene glycol, and injectable organic esters such as ethyl oleate can also be used. Other useful formulations include suspensions containing viscosity enhancing agents, such as sodium carboxymethylcellulose, sorbitol, or dextran. Excipients can also contain minor amounts of additives, such as substances that enhance isotonicity and chemical stability. Examples of buffers include phosphate buffer, bicarbonate buffer and Tris buffer, while examples of preservatives include thimerosol, cresols, formalin and benzyl alcohol.

Pharmaceutical carriers are known to those skilled in the art. These most typically would be standard carriers for administration to humans, including solutions such as sterile water, saline, and buffered solutions at physiological pH.

Molecules intended for pharmaceutical delivery can be formulated in a pharmaceutical composition. Pharmaceutical compositions can include carriers, thickeners, diluents, buffers, preservatives, surface active agents and the like in addition to the molecule of choice. Pharmaceutical compositions can also include one or more active ingredients such as antimicrobial agents, antiinflammatory agents, anesthetics, and the like.

The pharmaceutical composition can be administered in a number of ways depending on whether local or systemic treatment is desired, and on the area to be treated. Administration can be topically (including ophthalmically, vaginally, rectally, intranasally). In the case of contacting cells with the droplets described herein, it is possible to contact the cells in vivo or ex vivo.

Preparations for administration include sterile aqueous or non-aqueous solutions, suspensions, and emulsions. Examples of non-aqueous carriers include water, alcoholic/aqueous solutions, emulsions or suspensions, including saline and buffered media. Parenteral vehicles, if needed for collateral use of the disclosed compositions and methods, include sodium chloride solution, Ringer's dextrose, dextrose and sodium chloride, lactated Ringer's, or fixed oils. Intravenous vehicles, if needed for collateral use of the disclosed compositions and methods, include fluid and nutrient replenishers, electrolyte replenishers (such as those based on Ringer's dextrose), and the like. Preservatives and other additives can also be present such as, for example, antimicrobials, anti-oxidants, chelating agents, and inert gases and the like.

Formulations for topical administration can include ointments, lotions, creams, gels, drops, suppositories, sprays, liquids and powders. Conventional pharmaceutical carriers, aqueous, powder or oily bases, thickeners and the like can be necessary or desirable.

Dosing is dependent on severity and responsiveness of the condition to be treated. In one embodiment, the nanoemulsions described herein can be administered once a day for several days. Alternatively, the nanoemulsions can be adminstered once a week, once every two weeks, or once every three weeks. Persons of ordinary skill can easily determine optimum dosages, dosing methodologies and repetition rates. It is understood that any given particular aspect of the disclosed compositions and methods can be easily compared to the specific examples and embodiments disclosed herein based reagents discussed in the Examples. By performing such a comparison, the relative efficacy of each particular embodiment can be easily determined Particularly preferred compositions and methods are disclosed in the Examples herein, and it is understood that these compositions and methods, while not necessarily limiting, can be performed with any of the compositions and methods disclosed herein.

### EXAMPLES

The following examples are put forth so as to provide those of ordinary skill in the art with a complete disclosure and description of how the compounds, compositions, and methods described and claimed herein are made and evaluated, and are intended to be purely exemplary and are not intended to limit the scope of what the inventors regard as their invention. Efforts have been made to ensure accuracy with respect to numbers (e.g., amounts, temperature, etc.) but some errors and deviations should be accounted for. Unless indicated otherwise, parts are parts by weight, temperature is in ° C. or is at ambient temperature, and pressure is at or near atmospheric. There are numerous variations and combinations of reaction conditions, e.g., component concentrations, desired solvents, solvent mixtures, temperatures, pressures and other reaction ranges and conditions that can be used to optimize the product purity and yield obtained from the described process. Only reasonable and routine experimentation will be required to optimize such process conditions.

### Materials and Methods

### Drug.

Paclitaxel (PTX) was used as a chemotherapeutic agent. PTX was obtained from LC Laboratories (Woburn, Mass., USA).

### Preparation of Paclitaxel-Loaded Perfluoro-15-Crown-5 Ether (PFCE) Nanodroplets.

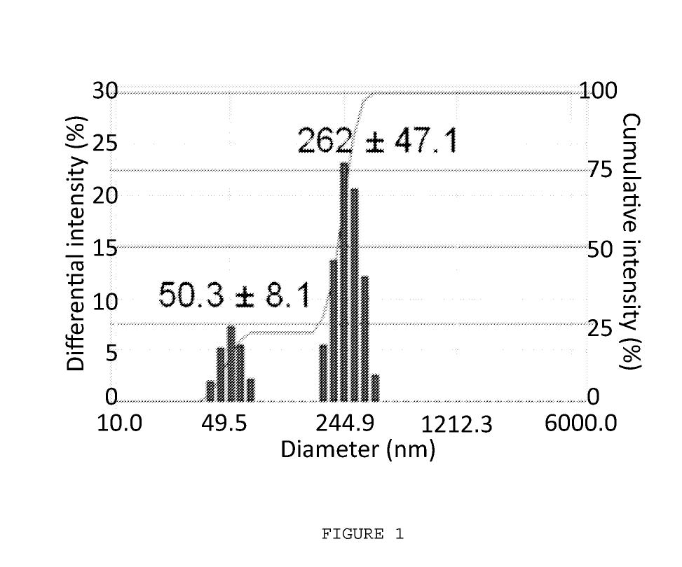

PTX-loaded perfluorocarbon nanodroplets were manufactured from PTX-loaded micelles formed by the water soluble, biodegradable block copolymer poly(ethylene oxide)-co-poly(D,L-Lactide) (PEG-PDLA) with molecular weight of either block of 2000 Da (Akina, Inc., West Lafayette, Ind., USA). PTX-containing PEG-PLA micellar solutions were prepared by a solid dispersion technique (Rapoport N, Efros A E, Christensen D, Kennedy A, Nam K-H. Microbubble Generation in Phase-Shift Nanoemulsions used as Anticancer Drug Carriers. Bub Sci Eng Tech 2009 1(1-2):31-39). Typically, 20 mg or 50 mg PEG-PDLA and 5 mg PTX were co-dissolved in 1 ml tetrahydrofuran (THF). The THF was then evaporated under gentle nitrogen stream at 60° C. or pumped out at room temperature. PTX-loaded micelles were reconstituted by dissolving residual gel matrix in 1 ml phosphate buffered saline (PBS, pH 7.4). Then 10 μl PFCE (MW 580.01, Oakwoods Products, Inc., West Columbia, S.C., USA) was introduced into micellar solution and emulsified by sonication on ice (VCX500, Sonics and Materials, Inc., CT, USA) to obtain paclitaxel-loaded droplets of the composition 2% or 5% PEG-PDLA/0.5% PTX/1% PFCE. The components of micellar or nanodroplet formulations were obtained from commercial sources and used without further purification. Micellar solutions and perfluorocarbon compounds were sterilized by filtration and mixed in a sterile test tube before being sonicated on ice for the generation of the nanoemulsion. The size of PFCE nanodroplets (both empty and drug loaded) was in the range 250 nm to 300 nm (FIG. 1).

### Subcutaneous PDA MiaPaCa-2 Tumor Model.

Human pancreatic cancer MiaPaCa-2 cells were obtained from the American Type Culture Collection (Rockville, Md., USA) and transfected with red fluorescence protein (RFP). Because only live cells generate RFP and therefore are fluorescent, the intravital whole mouse fluorescence imaging allowed the monitoring of tumor size and death of clusters of tumor cells. The excitation and emission peaks for the RFP were 563 nm and 587 nm, respectively.

Cells were maintained in DMEM media supplemented with 10% heat-inactivated fetal bovine serum (Gibco, Grand Island, N.Y., USA) at 37° C. in a 5% CO₂ incubator. Male nude mice between 6-8 weeks of age were utilized (NCr—Nu/Nu, National Cancer Institute, Frederick, Md., USA). For the tumor induction, mice were anesthetized with isoflurane and received a single subcutaneous injection of 1.5×10⁶ MiaPaCa-2 cells suspended in 150 μL of serum free DMEM. Tumors were grown in either the shoulder or thigh region and allowed to progress until reaching an initial size of at least 175 mm³, at which point mice were randomly assigned to a treatment group. Since the tumor size of untreated animals roughly doubles every week, the target initial tumor size of 175 mm³ was often exceeded and in the majority of animals the initial tumor size was 200-300 mm³ immediately before treatment. To assess the effect of the initial tumor size on treatment outcome, tumors were allowed to grow to the volume of roughly 1000 mm³ in a small subset of animals. All experiments were approved by the University of Utah Institutional Animal Care and Use Committee.

### MRgFUS Treatments.

At an assigned time point (2 hours or 8 hours) before magnetic resonance-guided FUS (MRgFUS) therapy, mice were systemically injected through the tail vein with either empty (i.e. non PTX-loaded) or PTX-loaded PFCE nanodroplets (PTX dose 40 mg/kg). In one experiment, the MRgFUS treatment was performed 10 minutes before the drug injection.

The MRgFUS treatments were executed with a small animal MRgFUS system (Image Guided Therapy, Inc., Pessac, France) with a 16-element annular transducer (f=3 MHz, r_{c}=3.5 cm, FWHM=1×3 mm) that could be translated in plane with piezo-ceramic motors. The system was placed in a Siemens 3T Trio scanner and temperature imaging was obtained using a 2D segmented-EPI sequence (TR/TE=60/10 ms, FA=15°, EPI=3, fat saturation, 752 Hz/pixel, 1.4 s acquisition, 2×2×3 mm³ resolution, single slice). Temperatures were reconstructed using a referenceless algorithm (Korc M. Pancreatic cancer-associated stroma production. Am J Surg 2007; 194(4 Suppl):584-86). and post-processed with zero-filled interpolation to yield 1×1×3 mm³ voxel spacing De La O J, Emerson L L, Goodman J L, Froebe S C, Ilium B E, Curtis A B, Murtaugh L C. Notch and Kras reprogram pancreatic acinar cells to ductal intraepithelial neoplasia. Proceedings of the National Academy of Sciences of the United States of America 2008; 105(48):18907-18912).

Four different acoustic peak pressure levels were applied: 2.4, 3.4, 4.2 and 4.8 MPa. These levels were calculated in water at the site of sonication assuming that the beam intensity is distributed evenly over the focal spot. Most experiments in the PTX-nanodroplet+MRgFUS group were performed with continuous wave (CW) ultrasound (N=24); in parallel with CW experiments, some experiments were performed with pulsed ultrasound (N=4) with pressure levels matching those of CW counterparts.

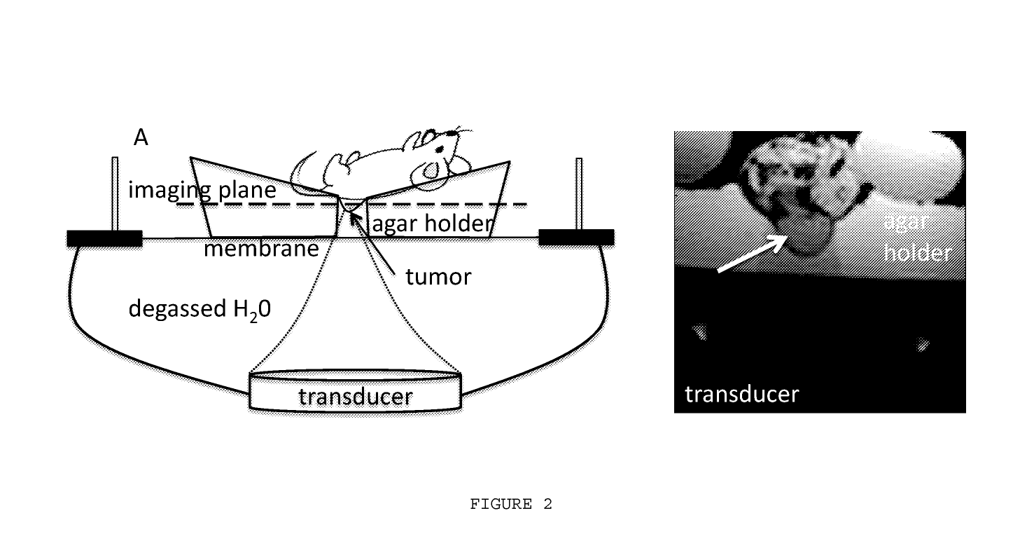

Treatments were conducted using the following protocol. The free-breathing anesthetized mouse (ketamine 100 mg/kg, xylazine 20 mg/kg) was placed on the agar holder such that the tumor protruded through the hole as shown schematically in FIG. 2A. In order to ensure an adequate acoustic window, the animal was always positioned with the tumor directly above the ultrasound transducer. An axial image of a mouse on the MRgFUS device placed in the magnet is shown in FIG. 2B. The transducer and agar holder are shown. The mouse is flanked by warm water filled tubes to help regulate its body temperature during the treatment.

The tumor was localized with high resolution sagittal and axial images. Temperatures were monitored in a single coronal slice placed at the focal plane of the transducer. Transducer speed was 1 and 2.5 mm/s in the 5 and 8 mm diameter spiral patterns, respectively and 0.1 mm/s in the grid pattern.

### Monitoring Treatment Outcome.

Time lines of tumor growth or regression were documented using three complimentary monitoring techniques: tumor size measurements with a caliper; tumor fluorescence imaging; and photography. All three techniques produced similar results. Fluorescence imaging allowed the monitoring of both tumor growth/regression and cell death in the tumor tissue (see below).

Tumor volume was calculated using the following equation:

V=(L×W²)/2 (1)

where L is the tumor length and W is the tumor width.

The end point corresponded to the tumor reaching about 2 cm in diameter; the time to reach this point was taken as a life span.

### Animal Groups.

The treatment groups and number of animals used in the study are listed in Table 1. Experimental parameters included PTX-loaded vs. empty nanodroplets, applied MRgFUS pressure and time of ultrasound application.

### Statistical Treatment.

The statistical significance of the differences between the pairs of groups were calculated using two-tail, two sample equal variance T-test; the differences were considered statistically significant for p<0.05.

### Results

Effect of the Combined Treatment with PTX-Loaded Nanodroplets and MRgFUS on Tumor Growth/Regression and Mouse Life Span.

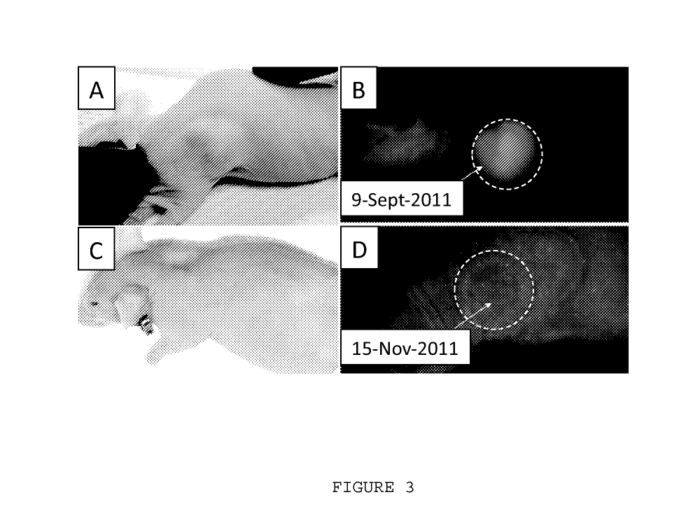

Two different scenarios of the tumor response to treatment were observed. The first involved a complete tumor resolution without recurrence. This was observed in four mice after a single treatment with PTX-loaded nanodroplets and CW MRgFUS at an acoustic peak pressure of 2.4 or 3.4 MPa for either a spiral or grid beam trajectory. An example of a complete tumor resolution with both photograph and fluorescence images is presented in FIG. 3.

The initial tumor was small (Vo=164 mm³); after the treatment, the tumor regressed quickly and there was no tumor visible to eye or by RFP imaging. However tumor re-growth started 6 weeks after the treatment. The recurrent tumor responded to a second treatment (nine injections of PTX-loaded nanodroplets without MRgFUS, twice a week for 4.5 weeks) indicating that tumor cells did not develop drug resistance. Histological examination of a control tumor showed the presence of mitotic cells and a pronounced stroma. In a recurred tumor in a mouse that received the combined treatment of PTX-nanodroplets and MRgFUS, no evidence of stroma and substantial necrosis in the residual tumor areas was observed. The presence of significant hemosiderin depositions in a treated tumor is a sign of the infarction of the primary treated tumor that appeared completely resolved and replaced with the scar tissue.

For a different mouse with a larger initial tumor (Vo=264 mm³) treated with the same protocol, complete tumor resolution took a longer time (eight weeks). The tumor did not recur during a five-months observation.

The cases of complete resolution of pancreatic cancer occurred after a single pancreatic tumor treatment with PTX-loaded nanodroplets and MRgFUS. This was observed in four of twenty-eight mice treated with PTX-loaded nanodroplets with various MRgFUS parameters. Survivors were observed at ultrasound acoustic pressures of 2.4 (N=1) or 3.4 MPa (N=3) and did not depend on the beam steering pattern (i.e. spiral or grid).

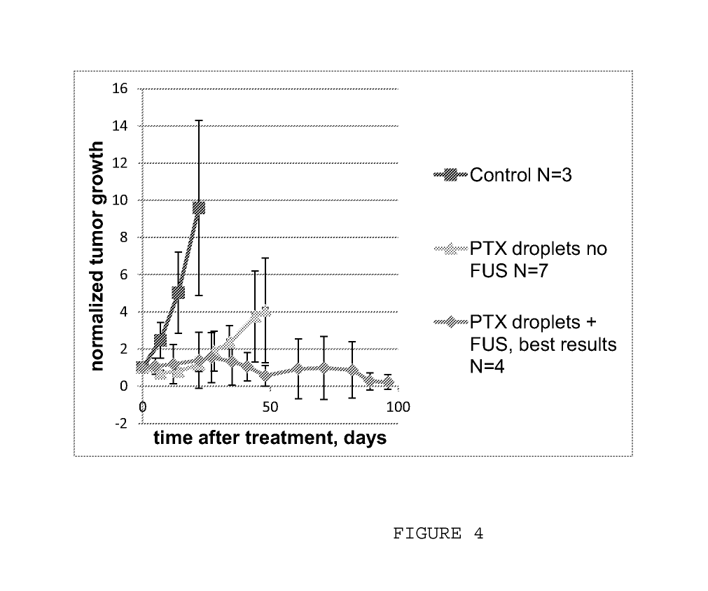

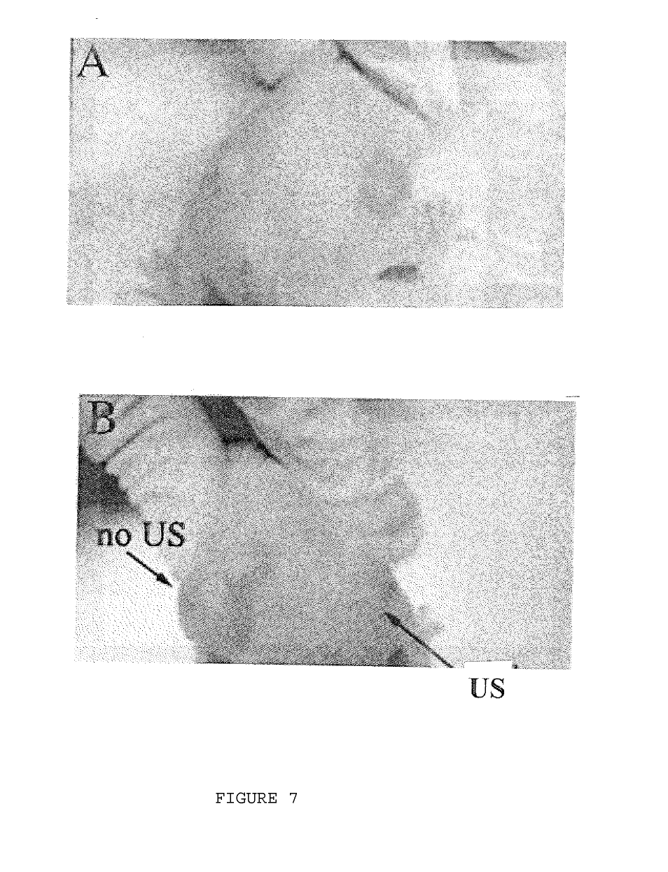

A therapeutic effect of PTX-loaded nanodroplets was also observed without the MRgFUS treatment (FIG. 4) though after a single treatment, the effect after a single treatment was stronger with ultrasound. Conversely, the use of nanoemulsions composed of PEG-PLLA requires the application ultrasound in order to reduce tumor growth. Referring to FIG. 7, when a mouse bearing two ovarian carcinoma tumors is administered a nanoemulsion composed of PEG-PLLA and PTX, tumor growth is not reduced after three weeks in the absence of ultrasound (left tumor in FIG. 7A). Only after ultrasound is applied to the tumor is there a reduction in tumor growth (right tumor in FIG. 7B). These results are presented in Rapoport et al. (J. Controlled Release 138, 2009, 268-276) Conversely, as shown in FIG. 4, tumor growth of pancreatic tumors is reduced when nanoemulsions composed of PEG-PDLA and PTX are adminstered to mice in the absence of ultrasound. Not wishing to be bound by theory, the nanoemulsion composed of PEG-PDLA biodegrades much faster when compared to PEG-PLLA. Thus, nanoemulsions composed of PEG-PDLA do not require ultrasound to release the therapeutic agent upon administration to the subject.

### The Role of Drug in the MRgFUS-Mediated Tumor Treatment: Comparison of the Effects of Empty and Drug-Loaded Nanodroplets.

Dramatic differences were observed in the tumor responses to MRgFUS treatment with and without drug. The MRgFUS tumor treatment without any injection did not affect tumor growth or mouse life span; any differences with control were not statistically significant.

Injections of empty (i.e. not PTX-loaded) nanodroplets without MRgFUS application or with MRgFUS pressure levels below 4.2 MPa did not exert any effect on the tumor growth rates or average mouse life span. Six mice were treated with empty nanodroplets with various MRgFUS pressure levels from 2.4 to 4.2 MPa; their average life span was 3.5 weeks, similar to negative control; however all mice treated with a pressure of 4.2 MPa died within one to three weeks after the treatment. In two cases, tumor growth was noticeably accelerated (data not shown). In contrast, the average life span of mice treated with PTX-loaded nanodroplets and MRgFUS was three-fold longer (10.3 weeks). These data indicate that for the combined PTX-loaded nanodroplets/MRgFUS treatment, the main therapeutic effect was caused by the drug and not by ultrasound. Still, as follows from FIG. 4 and Table 1, MRgFUS did enhance the action of the drug for certain combinations of ultrasound parameters.

### Effect of the Ultrasound Pressure.

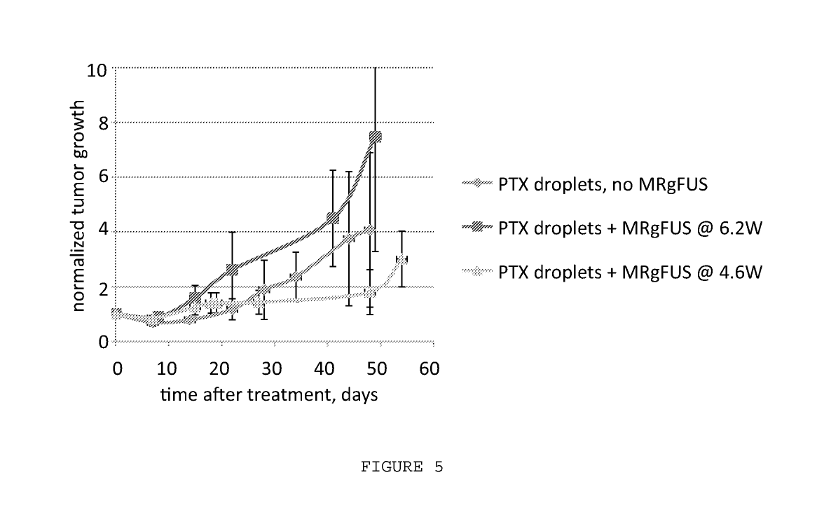

For mice treated with PTX-loaded nanodroplets and MRgFUS, increasing ultrasound pressure above 4.2 MPa exerted a detrimental effect on the tumor growth and animal survival (FIG. 5); moreover, at a pressure levels of 4.2 MPa and especially 4.8 MPa, grid-shaped skin burns that required special treatment were observed. The burns were resolved within two to three weeks.

### Effect of the Time of Ultrasound Application.

Experiments were performed with ultrasound application either two (N=5) or 6 to 8 hours after the injection (N=23). In one experiment, ultrasound was applied 10 minutes before the injection of PTX-loaded nanodroplets. No effect of MRgFUS was observed when ultrasound was applied either before or two hours after the nanodroplet injection; tumor growth rates and average life span did not differ from those observed for PTX-loaded nanodroplets without MRgFUS.

### Effect of Pulsed Ultrasound.

The experiments for pulsed and CW parameters were performed in parallel. The average life span of mice treated with pulsed ultrasound with various FUS parameters (6±1.4 weeks, N=4) was significantly lower than that of mice treated with CW ultrasound (10.3±1.6 weeks, N=19).

### Effect of the Initial Tumor Size.

The effect of the therapy depended strongly on the initial tumor size at the start of treatment. When the initial tumor size exceeded 1,000 mm³, the combined treatment by PTX-loaded nanodroplets and MRgFUS could not completely stop tumor growth; after the initial decrease of the tumor size, tumor growth resumed in three to four weeks. The average life span of animals with large initial tumors was increased by the treatment (roughly from 3 weeks for controls to 6 to 8 weeks for treated animals) but all tumors continued to grow despite the treatment. Increasing the MRgFUS treated volume by the treatment of the two tumor planes rather than one plane did not exert any positive effect on the life span of animals with large initial tumors. Moreover, tumor growth was accelerated after the two-plane treatment, presumably due to the increased heating (see discussion).

### Safety Issues and Collateral Damage.

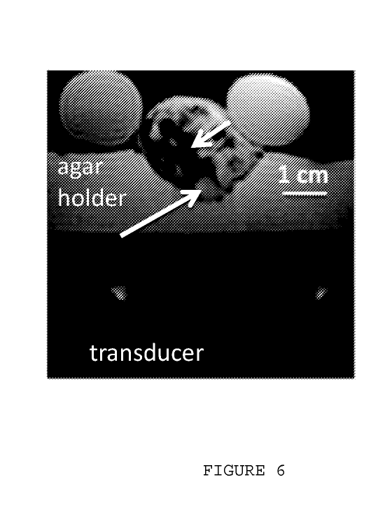

Seven of the total of fifty one animals (14%) treated with MRgFUS died within several days of the MRgFUS treatment. Four of seven animals died after the treatment with empty nanodroplets and MRgFUS at pressure levels of 4.2 or 4.8 MPa; two animals died after the treatment with the same MRgFUS parameters without any injection. One animal died two days after the combined treatment with PTX-loaded nanodroplets and MRgFUS at 4.2 MPa. No animal deaths resulted from the nanodroplet treatment without MRgFUS indicating that animal deaths were related to the MRgFUS treatment. Presence of empty nanodroplets during MRgFUS treatment appeared to increase the death rate. Although the exact mechanism that led to the animal's death is unknown, it is suspected that it may be due to peritonitis. A post-treatment analysis of MR images of coronal slices of MRgFUS treated animals suggested that the collateral damage occurred when gas-filled intestines were located in the far field of the ultrasound beam (FIG. 6).

TABLE 1
Treatment parameters for all animals used in the study

| | | MRgHIFU parameters | | | | Time | |
|---|---|---|---|---|---|---|---|
| | | | | Duty | | between | |
| | | | Acoustic | Cycle (%), | Total | injection & | Life |
| | Treatment | | Pressure | pulse | Sonication | MRgFUS | Span |
| N | Group | Trajectory | (MPa) | length | Time (s) | (hrs) | (weeks) |
| 7 | Negative Control | n/a | n/a | n/a | n/a | n/a | 3.5 ± 0.5 |
| | (No injection, no | | | | | | |
| | MRgFUS) | | | | | | |
| 7 | PTX- | | | | | | 7.0 ± 0.8 |
| | nanodroplets + no | | | | | | |
| | MRgFUS | | | | | | |
| 1 | No injection + | spiral | 3.4 | 100 | 240 | | 4.8 ± 2.3 |
| 1 | MRgFUS | | 4.2 | | 60 | | |
| 4 | | grid | 3.4 | | 325 ± 29 | | |
| 4 | | | 4.2 | | 300 | | |
| 3 | Empty | n/a | n/a | n/a | n/a | 6-8 | 3.5 ± 0.5 |
| | nanodroplets + | | | | | | |
| | MRgFUS | | | | | | |
| 1 | Empty | spiral | 3.4 | 100 | 60 | 6-8 | 3.5 ± 2.1 |
| 2 | nanodroplets + | | 4.2 | | 60 | | |
| 1 | MRgFUS | | 4.8 | | 240 | | |
| 4 | | grid | 3.4 | | 313 ± 25 | | |
| 4 | PTX- | grid | 3.4 | 100 | 300 | 1-2 | 7.0 ± 1.0 |
| 1 | nanodroplets + | | 2.4 | | | | |
| 4 | MRgFUS | spiral | 3.4 | 100 | 153 ± 54 | 6-8 | 10.3 ± 1.6 |
| 4 | | | 2.4 | 100 | 130 ± 50 | | |
| 1 | | | 4.2 | 100 | 60 | | |
| 1 | | | 4.8 | 100 | 60 | | |
| 5 | | grid | 3.4 | 100 | 300 | | |
| 2 | | | 4.8 | 100 | 300 | | |
| 2 | | | 4.2 | 100 | 300 | | |
| 1 | | grid | 3.4 | 50 (50 ms) | 300 | | 6.0 ± 1.4 |
| 1 | | grid | 4.2 | 50 (50 ms) | 300 | | |
| 1 | | spiral | 3.4 | 50 (100 ms) | 300 | | |
| 1 | | spiral | 4.8 | 50 (50 ms) | 300 | | |

*Mean values plus/minus standard deviations are presented.
**Mice that died within several days after the treatment (observed for the MRgFUS pressure of or higher than 4.6 W) were excluded from the life span calculation.
***Survivors (N = 2 for the grid trajectory) were excluded from the life span calculation.

Various modifications and variations can be made to the compounds, compositions and methods described herein. Other aspects of the compounds, compositions and methods described herein will be apparent from consideration of the specification and practice of the compounds, compositions and methods disclosed herein. It is intended that the specification and examples be considered as exemplary.

What is claimed:

1. A method for treating a tumor comprising contacting the tumor with a nanoemulsion comprising (1) at least one perfluoro crown ether and (2) a block copolymer comprising a hydrophilic block and hydrophobic block, wherein the hydrophobic block is poly(d,l)lactic acid, and wherein the nanoemulsion comprises a therapeutic agent encapsulated in the nanoemulsion, wherein the tumor is not exposed to ultrasonic radiation.

2. The method of claim 1, wherein the perfluoro crown ether has at least 16 fluorine atoms.

3. The method of claim 1, wherein the perfluoro crown ether is perfluoro 12-crown-4 ether, perfluoro 15-crown-5 ether, perfluoro 18-crown-6 ether, perfluoro 20-crown-7 ether, perfluoro dibenzo-18-crown-6 ether, perfluoro diaza-18-crown-6 ether, or any combination thereof.

4. The method of claim 1, wherein the perfluoro crown ether is perfluoro 15-crown-5 ether.

5. The method of claim 1, wherein the hydrophilic block of the block copolymer comprises a poly(alkylene oxide).

6. The method of claim 5, wherein the poly(alkylene oxide) comprises a polyethylene oxide, a polypropylene oxide, a polybutylene oxide, a polypentylene oxide, or a combination thereof.

7. The method of claim 5, wherein the poly(alkylene oxide) is polyethylene oxide.

8. The method of claim 5, wherein the poly(alkylene oxide) has a molecular weight of 500 to 10,000 Da.

9. The method of claim 5, wherein the poly(alkylene oxide) has a molecular weight of 1,500 to 2,500 Da.

10. The method of claim 1, wherein the a poly(d,l)lactic acid has a molecular weight of 500 to 12,000 Da.

11. The method of claim 1, wherein the a poly(d,l)lactic acid has a molecular weight of 1,500 to 2,500 Da.

12. The method of claim 1, wherein the therapeutic agent comprises a chemotherapeutic drug.

13. The method of claim 1, wherein the therapeutic agent is paclitaxel, doxorubicin, gemcitabine, adriamycin, cisplatin, taxol, methotrexate, 5-fluorouracil, betulinic acid, amphotericin B, diazepam, nystatin, propofol, testosterone, estrogen, prednisolone, prednisone, 2,3 mercaptopropanol, progesterone, or any combination thereof.

14. The method of claim 1, wherein the therapeutic agent is paclitaxel.

15. The method of claim 1, wherein the nanoemulsion has a diameter of 10 nm to 500 nm.

16. The method of claim 1, wherein the block copolymer is poly(ethylene oxide)-co-poly(d,l-lactide), the perfluoro crown ether is perfluoro 15-crown-5 ether, and the therapeutic agent is paclitaxel.

17. The method of claim 1, wherein the tumor or cancer is breast cancer, pancreatic cancer, ovarian cancer, prostate cancer, or colon cancer.

18. The method of claim 1, further comprising imaging the tumor by ¹⁹F MRI.

19. The method of claim 1, wherein the nanoemulsion is administered systemically to the subject by injection.

20. A method for treating cancer in a subject comprising administering to the subject having a tumor a nanoemulsion comprising (1) at least one perfluoro crown ether and (2) a block copolymer comprising a hydrophilic block and hydrophobic block, wherein the hydrophobic block is poly(d,l)lactic acid, and wherein the nanoemulsion comprises a therapeutic agent encapsulated in the nanoemulsion, wherein the tumor is not exposed to ultrasonic radiation.
